(19) United States
(12) Patent Application Publication
(10) Pub. No.: US 2002/0123525 A1
(43) Pub. Date: Sep. 5, 2002

(54) Cardioprotective composition and uses thereof

(75) Inventors: Mircea Alexandru Mateescu, Montreal (CA); Joanne Paquin, Montreal (CA); Denis Langlois, Ste-Anne des Lacs (CA); Xintao Wang, Bronx, NY (US); Mhamed Aouffen, Montreal (CA)

Correspondence Address: DARBY & DARBY P.C., 805 Third Avenue, New York, NY, 10022, US

(73) Assignee: GESTILAB INC., Ste-Anne des Lacs (CA)

(21) Appl. No.: 10/012,702

(22) Filed: Nov. 5, 2001

Related U.S. Application Data
(63) Continuation of application No. PCT/CA00/00530, filed on May 5, 2000.

(30) Foreign Application Priority Data
May 5, 1999 (CA) 2,271,193

(51) Int. Cl. A61K 31/19

(52) U.S. Cl. 514/557; 514/547

(57) ABSTRACT

A cardioprotective composition for protecting heart against oxidative stress and methods for using and preparing the same. More particularly, the cardioprotective composition of the invention comprises a mixture of pyruvate, antioxidant, and lipid(s) such as fatty acids. The cardioprotective composition could be used for the treatment of heart attack/failure, the treatment of ischemic cardiopathy, the conservation of heart before and during transplantation, and the treatment heart oxidative stress related conditions.

### BACKGROUND OF THE INVENTION

1. Field of the Invention

The present invention relates to the use of an amphiphilic composition as a cardioprotective agent and to methods for using and preparing the same. More particularly, the present invention pertains to the use of a formulation of pyruvate, antioxidant, and lipid(s) such as fatty acids for protecting heart against oxidative stress.

2. Description of the Prior Art

Reactive oxygen species (ROS) have been shown to be implicated in the development of many heart dysfunctions and ischemia/reperfusion insults to this organ are among the leading causes of mortality in America. These insults are caused by complete or partial local occlusions of vasculature and by trauma to heart, and also occur during handling of graft destined to heart surgery. Furthermore, evidence has been accumulated that oxygen free radicals (OFR) are, at least in part, responsible for specific damages and arrhythmias at reperfusion of ischemic heart. Therefore, lipid peroxidation of myocardial membranes by OFR has been considered a potential mechanism of reperfusion arrhythmias. Interestingly, many studies have shown that inhibition of free radical accumulation during myocardial ischemia and reperfusion with OFR scavengers, antioxidant enzymes and spin-trap agents reduce the severity of reperfusion-induced arrhythmias.

Until now, no ideal therapeutic agent was known to protect heart against oxidant species associated with various types of oxidative stress and, at the same time, to present good antifibrillatory action and with less side effects in arrhythmias associated with the reperfusion of ischemic heart. For instance, heparin presents antioxidant an antifribrillatory actions, but exhibits hemorrhagic side effects which limit its use.

TRIAD is a combination of pyruvate, antioxidant and fatty acids. This composition has been patented in 1997 in the U.S. as a therapeutic wound healing compositions (U.S. Pat. No. 5,652,274). Many related U.S. patents have also been issued for covering the uses of TRIAD in antikeratolytic compositions (U.S. Pat. No. 5,641,814); in anti-fungal compositions (U.S. Pat. No. 5,663,208); in acne healing compositions (U.S. Pat. No. 5,646,190); in anti-inflammatory compositions (U.S. Pat. No. 5,648,380); in dermatological compositions (U.S. Pat. No. 5,602,183); in sunscreen compositions (U.S. Pat. No. 5,674,912); in antihistamine compositions (U.S. Pat. No. 5,614,561); in cytoprotective compositions (U.S. Pat. No. 5,633,285); in wound healing composition affixed to razor cartridges (U.S. Pat. No. 5,682,302); and in regenerating compositions (EP 0 573 465 B1). However, none of these patents disclose or suggest the use of TRIAD as cardioprotective and antifibrillatory agent.

In view of the above, it is clear that there is a need for a partly lipidic and partly hydrophilic antioxidative composition comprising pyruvate, antioxidant, and lipid(s) such as fatty acids, to protect the heart against oxidant species and, at the same time, to provide important antifibrillatory effects in arrhythmias associated with the reperfusion of ischemic heart.

The purpose of this invention is to fulfil this need along with other needs that will be apparent to those skilled in the art upon reading the following specification.

### SUMMARY OF THE INVENTION

The present invention relates to a cardioprotective composition and more particularly to an amphiphilic antioxidative composition and its uses.

According to an aspect of the invention, the cardioprotective composition comprises a therapeutically effective amount of a mixture pyruvate, antioxidant(s), and lipid(s) such as fatty acids. These components are present in an amount that have a synergistic protective effect on cardiac cells.

In a preferred embodiment, lipids consist of a mixture of saturated and unsaturated fatty acids selected from the group consisting of monogylcerides, digylcerides, trigylcerides, free fatty acids, and mixtures thereof.

Preferably, pyruvate is selected from the group consisting of pyruvic acid, pharmaceutically acceptable salts of pyruvic acid, prodrugs of pyruvic acid, and mixtures thereof.

Preferably, also the antioxidant is selected from lipid-soluble antioxidants, and more preferably the antioxidant is selected from the group consisting of Vitamin A, carotene, Vitamin E, pharmaceutically acceptable salts thereof, and mixtures thereof.

According to an other aspect of the invention, the cardioprotective composition is used as such or as an active agent in the preparation of a medication for the treatment of heart and cardiac cells. Such treatments include the treatment of heart attack/failure, the treatment of ischemic cardiopathy, the conservation of heart before and during transplantation, and the treatment heart oxidative stress related conditions.

According to an other aspect of the invention, the invention provides a method for treating a heart oxidative stress related condition, the method comprising administrating to a patient in need thereof a therapeutically effective amount of an antioxidative composition comprising pyruvate, at least one antioxidant and at least one lipid.

Alternatively, the invention also provides a method for treating a heart oxidative stress related condition comprising: a) administrating to a patient in need thereof, a therapeutically effective amount of an antioxidative composition comprising pyruvate and at least one antioxidant; and b) providing, into the blood circulation of this patient, at least one lipid having a synergistic therapeutic effect on heart or cardiac cells with said antioxidative composition. The lipid(s) could be provided to the patient by increasing its lipidic blood level ratio through its diet. Examples of heart oxidative stress related conditions includes an heart attack/failure, ischemic cardiopathy, or handling an heart before and during an heart transplantation.

According to an other aspect of the invention it is provided a method for preparing a cardioprotective composition, the method comprising the steps of:

a) providing a therapeutically effective amount of: i) pyruvate, ii) at least one antioxidant; and iii) at least one lipid;

b) mixing together the components i), ii) and iii) of step a) in a physiological buffered saline solution to obtain a pharmaceutically acceptable homologous suspension; and optionally

c) centrifuging or filtering the homologous suspension obtained in step b).

The buffered saline solution may comprises sodium, potassium, magnesium and calcium ions at physiological concentrations and if necessary, an emulsifier.

An advantage of the present invention is that it provides effective means for preventing the loss of viability and/or stimulates the repair of cardiac cells in conditions of oxidative stress. It can also protect heart from a toxic substance or a stress, stabilizes the cellular membrane of a cardiac cell and/or helps in the normalization of cardiac cellular functions.

Other objects and advantages of the present invention will be apparent upon reading the following non-restrictive description of several preferred embodiments made with reference to the accompanying drawings.

### DESCRIPTION OF THE DRAWINGS

FIG. 1 is a diagram showing the time course protocol used for testing the composition of the invention.

FIG. 2 depicts in graphs the effect of various concentrations of TRIAD (o) or TRIAD (S2) (.) on LVP, and HR in isolated rat heart (n=4).

FIG. 3 depicts in bar graphs the effect of various concentrations of TRIAD (S2) on LVP, HR, and CF of isolated rat heart exposed to perfusion with electrolyzed buffer.

FIG. 4 is a graph showing the in vitro antioxidant capacity of TRIAD, TRIAD (S2) and pyruvate at various concentrations in conditions of electrolysis pro-oxidant system, evaluated by DPD method and expressed as ROS scavenging percentage.

FIG. 5A is a bar graph showing the incidence of irreversible fibrillation (IVF) in function of different TRIAD concentrations at reperfusion of ischemic isolated heart (n=4).

FIG. 5B is a bar graph showing the relation between different TRIAD concentrations with respect to the cardioprotection.

FIG. 6 shows ECG and LVP tracings with (+) and without (−) TRIAD protection.

### DETAILED DESCRIPTION OF THE INVENTION

As stated hereinbefore the present invention relates to the use of an amphiphilic antioxidative compositions as cardioprotective agent. As disclosed herein, a composition comprising sodium pyruvate, antioxidant and lipid(s) such as fatty acids have cardioprotective actions against oxidative stress.

Unless defined otherwise, all technical and scientific terms used herein have the same meaning as commonly understood by one ordinary skilled in the art to which this invention belongs.

As used herein, the term “cardioprotective agent” or “cardioprotective composition” refers to any compound (or to any mixture of compounds) that protects heart from a toxic substance or a stress, stabilizes the cellular membrane of a cardiac cell and/or helps in the normalization of cardiac cellular functions. As used herein, the terms “cardiac cells” includes cells from the organ (mainly myocytes) as well as endothelial vascular cells. A “cardioprotective agent” thereby prevents the loss of viability and/or stimulates repair of cardiac cells. It will also preferably improve, at the organ level, the cardiodynamic variables (coronary flow, heart rate, left ventricular pressure) of the heart in conditions of oxidative stress.

Therefore, the term “cardioprotection” as used herein refers to the capacity of a cardioprotective agent to maintain the cardiodynamic variables at their normal level or to induce a fast recovery to the normal level, even in pathological or harmful conditions such as oxidative stress conditions including those occurring at post-ischemia reperfusion and inflammation.

As stated out above, the cardioprotective composition of the invention comprises a mixture of (a) pyruvate, (b) an antioxidant, and (c) at least one lipid such as fatty acids, preferably a mixture of saturated and unsaturated fatty acids. According to the invention, these three components have a synergistic beneficial effect on cardiac cells, i.e. their combined effect is greater than the sum of their individual effects.

The pyruvate in the present invention may be selected from the group consisting of pyruvic acid, pharmaceutically acceptable salts of pyruvic acid, prodrugs of pyruvic acid, and mixtures thereof. In general, the pharmaceutically acceptable salts of pyruvic acid may be alkali salts and alkaline earth salts. Preferably, the pyruvate is selected from the group consisting of pyruvic acid, lithium pyruvate, sodium pyruvate, potassium pyruvate, magnesium pyruvate, calcium pyruvate, zinc pyruvate, manganese pyruvate, methyl pyruvate, α-ketoglutaric acid, and mixtures thereof. More preferably, the pyruvate is selected from the group of salts consisting of sodium pyruvate, potassium pyruvate, magnesium pyruvate, calcium pyruvate, zinc pyruvate, manganese pyruvate, and the like, and mixtures thereof. Most preferably, the pyruvate is sodium pyruvate.

The amount of pyruvate present in the cardioprotective composition of the present invention is a therapeutically effective amount. A therapeutically effective amount of pyruvate is that amount of pyruvate necessary for the cardioprotective composition to prevent and/or reduce injury of heart. The exact amount of pyruvate will vary according to factors such as the type of condition being treated as well as the other ingredients in the composition. Typically, the amount of pyruvate should vary from about 0.01 mM to about 100 mM. In a preferred embodiment, pyruvate is present in the composition of the cardioprotective perfusing solution in an amount from about 0.1 mM to about 20 mM, preferably from about 0.5 mM to about 10 mM. In the most preferred embodiment, the cardioprotective composition comprises about 2.5 mM of sodium pyruvate.

Antioxidants are substances which inhibit oxidation or suppress reactions promoted by oxygen, oxygen free radicals (OFR), oxygen reactive species (ORS) including peroxides. Antioxidants, especially lipid-soluble antioxidants, can be absorbed into the cellular membrane to neutralize oxygen radicals and thereby protect the membrane. The antioxidants useful in the present invention are preferably vitamin antioxidants that may be selected from the group consisting of all forms of Vitamin A including retinal and 3,4-didehydroretinal, all forms of carotene such as Alpha-carotene, β-carotene, gamma-carotene, delta-carotene, all forms of Vitamin C (D-ascorbic acid, L-ascorbic acid), all forms of tocopherol such as Vitamin E (Alpha-tocopherol, 3,4-dihydro-2,5,7,8-tetramethyl-2-(4,8,12-trimethyltri-decyl)-2H-1 -benzopyran-6-ol), β-tocopherol, gamma-tocopherol, delta-tocopherol, tocoquinone, tocotrienol, and Vitamin E esters which readily undergo hydrolysis to Vitamin E such as Vitamin E acetate and Vitamin E succinate, and pharmaceutically acceptable Vitamin E salts such as Vitamin E phosphate, prodrugs of Vitamin A, carotene, Vitamin C, and Vitamin E, pharmaceutically acceptable salts of Vitamin A, carotene, Vitamin C, and Vitamin E, and the like, and mixtures thereof. Preferably, the antioxidant is selected from the group of lipid-soluble antioxidants consisting of Vitamin A, β-carotene, Vitamin E, Vitamin E acetate, and mixtures thereof. More preferably, the antioxidant is Vitamin E or Vitamin E acetate. Most preferably, the antioxidant is Vitamin E acetate. Analogues of Vitamin E such as Trolox®, a compound which is more hydrosoluble than natural forms of Vitamin E and which could reach intracellular sites more rapidly, could also be used according to the present invention.

The amount of antioxidant present in the cardioprotective compositions of the present invention is a therapeutically effective amount. A therapeutically effective amount of antioxidant is that amount necessary for the cardioprotective composition to prevent and/or reduce injury of a heart or cardiac mammalian cells. The exact amount of antioxidant will vary according to factors such as the type of condition being treated as well as the other ingredients in the composition. Typically, the amount of antioxidant should vary from about 0.01 unit/ml to about 10 unit/ml. In a preferred embodiment, vitamin E antioxidant is present in the composition of the cardioprotective solution in an amount from about 0.01 unit/ml to about 2 unit/ml, preferably from about 0.05 unit/ml to about 1 unit/ml. In the most preferred embodiment, the cardioprotective composition comprises about 0.25 unit of antioxidant α-tocopherol type VI in oil) per ml of cardioprotective composition.

As it is well known, lipids are esters or carboxylic acid compounds found in animal and vegetable fats and oils. The composition may comprises a single type of lipid or various types of different lipids. Preferably lipids are in the form of a mixture of saturated and unsaturated fatty acids. However, other types of lipids could be used such as glycolipids and phospholipids (e.g. lecithin). Lipid(s) or mixture thereof are selected among those lipids required for the stabilization or repair of the cellular membrane of cardiac mammalian cells. These lipids may be derived from animal or vegetables. In a preferred embodiment, selected lipids are in the form of mono-, di-, or triglycerides, or free fatty acids, or mixtures thereof, which are readily available for the stabilization or repair of the cellular membrane of cardiac mammalian cells. Artificial lipids which are soluble in organic solvents and are of a structural type which includes fatty acids and their esters, cholesterols, cholesteryls esters could also be used according to the present invention.

In a more preferred embodiment, the saturated and unsaturated fatty acids are those deriving from egg yolk. According to the use of the cardioprotective composition of the invention, replacing egg yolk as a source of fatty acids by chemical preparations of unsaturated, polyunsaturated and/or saturated fatty acids compatible with, and in proportions similar to those found in cell membranes, may be advantageous or reveal necessary to insure a controllable quality of preparations.

The amount of lipid(s) such as fatty acids present in the cardioprotective composition of the present invention is a therapeutically effective amount. A therapeutically effective amount of fatty acids for instance is that amount of fatty acids necessary for the cardioprotective composition to prevent and/or reduce injury of a cardiac tissue, without being toxic to cardiac cells. The exact amount of lipid(s) or fatty acids will vary according to factors such as the type of condition being treated as well as the other ingredients in the composition. Typically, the amount of lipid(s) or fatty acids should vary from about 0.001% v/v to about 1% v/v. In a preferred embodiment, fatty acids are present in the composition of the cardioprotective perfusing solution in an amount from about 0.001% v/v to about 0.2% v/v, preferably from about 0.005% v/v to about 0.1% v/v, by weight of cardioprotective composition. In the most preferred embodiment, the cardioprotective composition comprises about 0.025% v/v of fresh egg yolk.

As the lipidic blood level of an individual is normally about 0.5-0.6% of the total serum volume, the lipidic portion could be omitted from the cardioprotective composition of the invention. It could be possible to provide into the blood circulation of this individual at least one lipid having a synergistic therapeutic effect on cardiac cells with the others component of the antioxidative cardioprotective composition of the invention. For instance, selected lipid(s) could be provided by increasing the lipidic blood level ratio of this individual through the diet. Lipids which could have a synergistic therapeutic effect without being harmful to a patient could be selected from the group consisting of phospholipids, glycolipids, fatty acids, and mixture thereof.

Further agents can be joint to the cardioprotective composition of the invention. For examples various antioxidants may complete the action of the cardioprotective composition such as:

ceruloplasmin or its analogues since it can scavenge ^{·}O₂⁻ radicals and has a ferroxidase activity which oxidizes Fe²⁺ to Fe³⁺;

metal chelators/scavengers (e.g. desferrioxamine [Desferal®], a small substance capable to scavenge Fe³⁺ and other metal ions);

proteins or their fragments that can bind metal ions such as ferritin, or transferrin which both bind Fe³⁺;

small scavengers Of ^{·}O₂⁻ (superoxide), ^{·}OH (hydroxyl) or NO (nitric oxide) radicals (e.g. acetyl salicylic acid, scavenger of ^{·}O₂⁻; mannitol or captopril, scavengers of ^{·}OH) or molecules that inhibit the generation of these radicals (e.g. arginine derivatives, inhibitors of nitric oxide synthase which produce NO);

proteins or their fragments that scavenge OFR and can assist the protective action of ceruloplasmin (e.g. superoxide dismutase which dismutate ^{·}O₂⁻; hemoglobin which traps NO); and

proteins or their fragments that can scavenge H₂O₂ (hydrogen peroxide) in cases where they may exert a more potent or durable protective action than pyruvate (e.g. catalase, glutathion peroxidase).

The composition of the invention may also comprises modulators of heart functions such as hormones, trophic factors, or analogues of these substances that act by binding to heart receptors (e.g. ligands of β-adrenergic receptors in cardiac arrhythmia.

Further to the therapeutic agents, the cardioprotective composition of the invention may also contain preserving agents, solubilizing agents, stabilizing agents, wetting agents, emulsifiers, sweeteners, colorants, odorants, salts, buffers, coating agents or antioxidants. For preparing the cardioprotective composition, methods well known in the art may be used.

The method of preparation of the cardioprotective composition of the invention consists simply in the mixing of components in a buffered saline solution in order to get a homogenous suspension. Suitable saline solution comprises sodium, potassium, magnesium and calcium ions at physiological concentrations, has an osmotic pressure varying from 280 to 340 mosmol, and a pH varying from 7.0 to 7.4. Depending of the amount and of type of lipid(s) which is used, the saline may also comprises an emulsifier. Preferably, the buffered saline solution is selected from the group consisting of modified Krebs-Henseleit buffer (KH) and phosphate buffer saline (PBS), both at pH 7.4. The homogenous suspension obtained can further be centrifuged and/or filtered to reduce its viscosity and/or eliminated non-soluble particles.

Obviously, this simple method can be modified according to the use of the cardioprotective composition. For instance, in the example found hereunder, genuine and centrifuged-filtered preparations were used. However, it is important to note that modifications in the modality of preparation can, in a certain extent, influence the resulting effects of the cardioprotective composition. For example, varying the pH of the composition (or buffer) can slightly modify the ionization state of carboxylic function of pyruvate and thus alter its solubility and/or reaction with H₂O₂ while the dialysis of the composition would reduce the amount of pyruvate in the final preparation, unless it is done before addition of pyruvate. A person skilled in the art will know how to adapt the preparation of the cardioprotective composition of the invention according to its uses in specific conditions in order to obtain positive desired effects.

The cardioprotective composition of the invention could be suitable to treat diseases and pathological conditions such as heart attack/failure and heart diseases (ischemic cardiopathy). The cardioprotective composition of the invention could also be used during the handling of organs in transplantation (conservation of organs before and during transplantation, post-surgery survival). The cardioprotective composition could also be involved in the treatment of diseases which were shown to involve oxidative stress conditions such as non-viral but drug related hepatitis, in the treatment of poisoning or the diminution of side effects of various drugs (such as chemotherapeutic and immunosuppressive drugs) since deleterious action of various toxicants and drugs is exerted via production of reactive oxygen species.

The cardioprotective composition of the invention has potential applications in both fast (in minutes; especially due to the pyruvate) and long term treatments (hours and days due to the antioxidant(s) and lipid(s) such as fatty acids). The amount to be administered is a therapeutically effective amount. A therapeutically effective amount of a cardioprotective composition is that amount necessary for protecting heart from a toxic substance, stabilizing the cellular membrane of cardiac cells and/or helping in the normalization of cardiac cellular and organ functions. Suitable dosages will vary, depending upon factors such as the type and the amount of each of the components in the composition, the desired effect (fast or long term), the disease or disorder to be treated, the route of administration and the age and weight of the individual to be treated.

The cardioprotective composition of the invention and/or more complex pharmaceutical compositions comprising the same may be given orally in the form of tablets, capsules, powders, syrups, etc. since all their components are absorbable by the gastrointestinal tract. Others administration ways can also be considered (rectal and vaginal capsules or nasally by means of a spray). They may also be formulated as creams or ointments for topical administration. They may also be given parenterally, for example intravenously, intramuscularly or sub-cutaneously by injection or by infusion. Intravenous administration can be a way for fast answer in various clinical conditions (e.g. stroke and heart attacks, post-surgery treatments, etc). Obviously, the cardioprotective compositions of the invention may be administered alone or as part of a more complex pharmaceutical composition according to the desired use and route of administration. Anyhow, for preparing such compositions, methods well known in the art may be used.

As it will now be demonstrated by way of an example hereinafter, the composition of the invention possesses a strong cardioprotective activity i.e. the capacity to maintain the cardiodynamic variables at their normal level or to induce a fast recovery to the normal level, even in pathological or harmful conditions such as oxidative stress conditions including those occurring at post-ischemia reperfusion inflammation. Although any methods and materials similar or equivalent to those described herein can be used in the practice or testing of the present invention, the preferred methods and materials are described.

### EXAMPLE

### Cardioprotective Actions of TRIAD Against Oxidative Stress Abstract

This work shows that TRIAD, a combination of sodium pyruvate, vitamin E and fatty acids, has an antioxidant protective action on isolated rat hearts exposed to oxidative stress. Two prooxidant situations were tested: 1) perfusion with electrolyzed buffer, and 2) partial ischemia followed by reperfusion. TRIAD induced resistance to injury caused by oxidative stress was assessed by evaluation of the ECG (electrocardiogram) profile and of cardiodynamic variables (Left Ventricular Pressure, Coronary Flow, Heart Frequency).

TRIAD concentrations equal or less than 1× permitted to achieve complete protection of hearts, and as low as 0.25× TRIAD was sufficient to protect hearts against injury induced by partial ischemia and reperfusion. Generally, in the experimental models, pyruvate was a major contributor of the antioxidant action of TRIAD and its effect was increased mostly in an additive manner and in some cases synergistically, by fatty acids and vitamin E.

Abbreviations CF: coronary flow; DPD: N,N-diethyl-p-phenylenediamine; ECG: electrocardiogram; HR: heart rate; KH: Krebs-Henseleit; LVP: left ventricular pressure; PBS: phosphate buffer saline; OFR: free oxygen radical; ROS: reactive oxygen species; SOD: superoxide dismutase; CAT: catalase; GP: glutathion (GSH)-peroxidase.

### 1. Introduction

1.1 Oxidative Stress and Antioxidant Defenses in Normal and Pathophysiological Heart and Brain

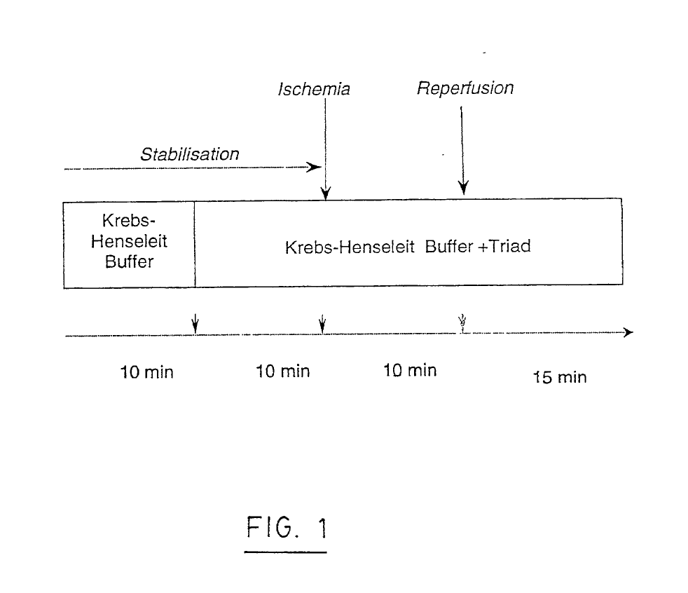

Reactive oxygen species (ROS) including hydrogen peroxide, free oxygen radicals (OFR) such as superoxide and hydroxyl radicals, and their derivatives are generated by normal cellular metabolism but are potent cellular toxicants when they are produced in excess and thus cause an oxidative stress to cells (LeBel and Bondy, 1991; Gutteridge, 1994; Chan, 1996). The organism has several strategies to maintain ROS-induced damage at low levels: a) to eliminate ROS (e.g. SOD, CAT and GP enzymes shown in FIG. 1), b) to scavenge ROS by trapping them (e.g. ascorbic acid) or by breaking their propagation (e.g. vitamin E), c) to sequester iron or other metals in non- or low reactive forms, and d) to repair molecular damages (Gutteridge, 1994).

ROS have been implicated in the development of many heart and brain dysfunctions (Takemura et al., 1994; Chan, 1996; Maiese, 1998) and ischemia/reperfusion insults to these organs are among the leading causes of mortality in America (Takemura et al., 1994; Chan, 1996; Maiese, 1998). These insults are caused by complete or partial local occlusions of vasculature and by trauma to heart and brain, and also occur during handling of grafts destined to heart surgery. After these ischemic events, when blood flow is restored (reperfusion), oxygen free radicals are released in situ as the main cause of oxidative damage.

1.2. Oxygen Free Radicals (OFR) and Reactive Oxygen Species (ROS) in Heart Arrhythmias

Evidence has been accumulated that OFR are, at least in part, responsible for specific damages and arrhythmias at reperfusion of ischemic heart (McCord, 1985). Various pathways generating superoxide radical (^{·}O²⁻) and other ROS—also known as reactive oxygen intermediates (ROI)—have been identified, such as: activation of polymorphonuclear leukocytes, autoxidation of catecholamines, reactions of xanthine oxidase and NADPH oxidase, or metabolism of arachidonic acid. The harmful effects of superoxide radical and its by-products are dramatically increased in the presence of transition metals. The ferrous (Fe²⁺) ion generated by the Haber-Weiss reaction catalyses the formation of the highly aggressive hydroxyl (^{·}OH) radical, via Fenton reaction (see section 4: Discussion). The presence of OFR has been measured in ischemic and reperfused myocardium directly by electron paramagnetic resonance spectroscopy and indirectly by biochemical assays of malondialdehyde (MDA) as an indicator of lipid peroxidation. The OFR concentration at reperfusion is higher than during ischemia. OFR may contribute to reperfusion injury by interacting with membrane polyunsaturated fatty acids (PUFA) and generating lipid peroxides which increase membrane permeability and alter ionic homeostasis. Lipid peroxidation of myocardial membranes by OFR, has been considered a potential mechanism of reperfusion arrhythmias.

Inhibition of free radical accumulation during myocardial ischemia and reperfusion with OFR scavengers, antioxidant enzymes, and spin-trap agents was shown to reduce the severity of reperfusion-induced arrhythmias in many studies.

It would be, therefore, highly desirable to obtain a therapeutic agent which would protect heart against oxidant species associated with various types of oxidative stress and at the same time, would present antifibrillatory effects in arrhythmias associated with the reperfusion of ischemic hearts. Such a therapeutic agent will be of a high utility, since it was recently shown that possibly, fibrillation generates OFR (Ferdinandy et al., 1993). There are several drugs used as antiarrhythmic agents, classified as per Vaughan Williams (1991) as: sodium channel blockers (e.g. quinidine, lidocaine, etc), β-blocking agents (propanolol), potassium channel blockers (amiodarone) and calcium channel blockers (verapramil, diltiazem, etc). TRIAD differs from these drugs and was not studied until now as antiarrhythmic agent on Langendorff isolated heart model. Therefore this study is the first showing that TRIAD has an antifibrillatory effect on heart ex vivo in addition to its cardioprotective action.

1.3 Aspects on TRIAD and Its Therapeutic Role

As stated hereinbefore, TRIAD is a combination of sodium pyruvate, antioxidant and fatty acids for which many uses have been patented. Preferably, TRIAD comprises sodium pyruvate, Vitamin E and egg yolk. Although this combination is also known under the name of CRT (Cellular Resuscitation Therapy), the current denomination of TRIAD is used throughout this document.

These three agents were shown to act synergistically to ameliorate wound healing (Martin, 1996; Sheridan et al., 1997) and to reduce oxidative damage to keratinocytes and monocytes exposed to ultraviolet light (Martin, 1996) or to hepatocytes treated with doxorubicin (Gokhale et al., 1997). The presumed respective role of each agent of the antioxidant combination is: a) for pyruvate, to bind stoichiometrically to H₂O₂, b) for vitamin E, to interrupt the propagation of lipid peroxidation, and c) for egg yolk, to provide a balanced mix of fresh unsaturated and saturated fatty acids which will help in membrane repair (Martin, 1996).

1.4 Presentation of the Study

The goal of this study was to determine if TRIAD has an antioxidant protective action on isolated rat hearts exposed to oxidative stress. The choice of this model is related to the fact that isolated rat heart in Langendorff montage is the most important experimental model in pharmacological evaluation of cardioprotective drugs. Two prooxidant situations were tested: perfusion with electrolyzed buffer and partial ischemia followed by reperfusion. Electrolysis is normally not a pathophysiological condition as is ischemia-reperfusion; however, it was used in this work since it generates several naturally-occurring ROS (Chahine et al., 1991), including ^{·}O₂⁻, H₂O₂, ^{·}OH, ¹O₂ (singlet oxygen) and, in addition, HOCI (hypochlorous acid) which is produced by activated macrophages in inflammation (Chahine et al., 1991). The protective action of TRIAD on hearts subjected to electrolysis-induced damage would also be directly comparable to that of ceruloplasmin for which a cardioprotective effect has been demonstrated in these conditions of stress (Chahine et al., 1991). TRIAD-induced resistance of heart to injury was assessed by measurement of cardiodynamic parameters: left ventricular pressure (LVP), heart rate (HR), coronary flow (CF), and electrocardiogram (ECG). In all cases, different concentrations of TRIAD were tested in order to determine those that permitted to achieve a complete protection and also tested the contribution of TRIAD components to the overall protection. In addition, when applicable, the antioxidant properties of TRIAD in vitro were measured in order to understand some aspects of the protection afforded by this mix in live models.

### 2. Materials and Methods

Materials

Vitamin E (α-tocopherol type VI in oil), sodium pyruvate, ethylenediamine tetraacetic acid (EDTA), and N,N-diethyl-p-phenylenediamine (DPD) were purchased from (Sigma Chem. Co.). Fresh egg yolk was the source of fatty acids. The other current chemicals were reagent grade (from Sigma Chem. Co., St-Louis) and were used without further purification.

Animals

Adult male Wistar rats (225-250 g) were from Charles River Inc. (Canada).

Methods

2.1 Preparation of TRIAD and TRIAD (S2)

The 1× TRIAD concentration was prepared as Gokhale et al. (1997) and contained 0.1% v/v fresh egg yolk, 1 unit/ml vitamin E (α-tocopherol type VI in oil) and 10 mM sodium pyruvate. Stock 5× (5 fold) or 10× (10 fold) concentration of TRIAD was freshly prepared before each experiment by carefully mixing the three agents to get a homogenous suspension. TRIAD mixtures were made in a modified Krebs-Henseleit (KH) buffer (118 mM NaCI, 25 mM NaHCO₃, 3.8 mM KCI, 1.2 mM KH₂PO₄, 1.2 mM MgSO₄, 2.5 mM CaCI₂, 11 mM dextrose, pH 7.4). Pyruvate was soluble in and egg yolk miscible with both saline physiological buffers.

It was found that TRIAD was not compatible with the organ functions (see section 3.1 of Results). Therefore the genuine TRIAD preparations were modified as follows: 5× or 10× genuine preparations were centrifuged at 15000×g for 20 min, at 4° C., and the resulting supernatants (S1) filtered on Whatman paper filter #54. The final filtered supernatant was named TRIAD (S2) and used to perfuse hearts. The different concentrations of TRIAD (S2) preparation were obtained by subsequent dilution with KH buffer (i.e. TRIAD (S2) 1× was obtained by 10 fold dilution of stock TRIAD (S2) 10× preparation). These supplementary steps yield in a less cloudy and less viscous preparations which were non toxic to the heart.

2.2 Isolated Heart Preparation and Perfusion Protocol

All experiments were conformed to rules of the Guide for the care and use of laboratory animals published by the U.S. National Institutes of Health (NIH publication No 85-23, revised 1985). Adult male Wistar rats (225-250 g) were anaesthetized with sodium pentobarbitone (0.1 ml/100 g body weight) and then heparinised (500 Ul intra-peritoneally). Hearts were rapidly excised, placed in ice-cold oxygenated KH buffer solution, cleaned and then mounted on a modified Langendorff heart perfusion apparatus.

Hearts were cannulated via the aorta and retrogradely perfused at a constant perfusion pressure (90 mm Hg at 37° C.) with modified KH buffer. This solution was continuously gassed with a mixture of 95% O2 and 5% CO2 to maintain a pH of 7.4 at 37° C. (with water jackets around the pressurized arterial reservoir by constant-temperature circulators). In order to avoid precipitates, the perfusion buffer was filtered through a 5.0 μm cellulose acetate membrane to remove particulate contaminants.

Recorded Cardiodynamic Indices

A saline-filled latex balloon was inserted into the left ventricle by way of the AV valve and connected via a polyethylene cannula to a pressure transducer for determination of Left Ventricular Pressure (LVP) and Left Ventricular End Diastolic Pressure (LVEDP). The intraballoon volume was adjusted to exert a physiologic LVEDP of 10 mm Hg. Epicardial electrogram (ECG) was obtained using two silver electrodes, one inserted into the ventricular apex, and the other connected to the aortic cannula. The LVP, LVEDP, and ECG were recorded on a Nihon-Kohden polygraph (RM 600); heart rate (HR) was calculated from the electrogram. Coronary flow (CF) was measured by time collection of coronary effluent at various times during the experiment.

The cardioprotective effect of TRIAD was investigated in two models: 1) in electrolysis induced ROS and 2) in reperfusion induced arrhythmias in partial (regional) ischemic isolated rat hearts.

2.3. TRIAD Cardioprotective Effects in Electrolysis Induced ROS on Isolated Rat Heart

After 10 min period of heart equilibration (Mateescu et al., 1995), the heart was submitted to electrolysis (Els) (10 mA DC generated by the Grass stimulator, for 1 min). The blank group of hearts (n=12) was without any treatment (no Els, nor TRIAD protection). The TRIAD was administered for a duration of 21 min covering 10 min before Els, the 1 min electrolysis and 10 min after. Electrolysis of perfusing KH buffer was realized as described by Jackson et al. (1986), by placing the two platinum wire electrodes in the inflow cannula above the heart. The anode was placed at 12 cm and the cathode at 15 cm from the left atrium. A glass bubble trap was placed above the aorta with the role to trap gas bubbling. Cardioprotection capacity was defined as the level of each cardiodynamic variable and was calculated as percentage of the value measured at different times, from the value of control groups.

Experimental groups studied:

1) A blank group of hearts (n=12), perfused without treatment and without electrolysis.

2) Treated groups, each of them (n=4) perfused with TRIAD preparations at different concentrations, without electrolysis (in order to rule out possible effects on the heart).

3) Control group (CTL), submitted to electrolysis without treatment (n=12).

4) Electrolysis-treated groups (n=4), each of them treated with TRIAD at a given concentration, and submitted to electrolysis.

The cardiodynamic variables were monitored during all the experimental period.

2.4. TRIAD Cardioprotective Effects on Isolated Rat Heart in Ischemia-reperfusion Model

Hearts were perfused for a 20 min control period with KH buffer, for stabilization. Regional ischemia was induced by occluding the left anterior descending artery with a tight ligature positioned around and at a point close to its origin, with a piece of plastic tubing. The resulting arterial occlusion that produces regional (partial) ischemia and consequently a reduction in coronary flow of 40%-50%, was maintained for 10 min. In fact, an acceptable regional ischemia was confirmed, in addition to the mentioned CF reduction, by 60-70% LVEDP elevation and by 40-50% LVP reduction. At the end of this 10 min arterial occlusion period, reperfusion was initiated by cutting the ligature with a scalpel bled and rhythm disturbances were monitored for 15 min more. Left ventricular pressure and epicardial ECG were continuously monitored before and during ischemia and reperfusion.

Several experimental groups were studied, according to the time course protocol depicted in FIG. 1. Hearts in the control group (n=12) were perfused with KH buffer throughout the experiment and submitted to 10 min partial ischemia without any cardioprotective (i.e. TRIAD) treatment. Concentration-effect relationship in cardioprotection were established by treatment of hearts in ischemia and reperfusion with different concentrations of TRIAD (0.1-2×) added to the KH perfusing buffer (n=4 for each TRIAD concentration). The treatment was initiated 10 min before ischemia and continued over the whole ischemia-reperfusion experiment. Thus, TRIAD was administrated 10 min before coronary occlusion, during the ischemia period, and 15 min of reperfusion period. Cardioprotective effects of TRIAD were compared with previous data on the antiarrhythmic effects of deferoxamine (500 μM)—an iron chelator produced by bacteria (Streptomyces pilosus) and of ceruloplasmin—a copper protein recently shown to exhibit an important antifibrillatory effect in ischemia-reperfusion (Atanasiu et al., 1995).

Quantification of Arrhythmias

Arrhythmias were defined according to the Lambeth convention (Walker et al., 1988). Electrograph recordings were analyzed for the incidence of irreversible ventricular fibrillations (IVF) and for the time of normal sinus. It was analyzed whether fibrillation was spontaneously reversible, or hearts remained in irreversible ventricular fibrillation (more than 120 seconds). Ventricular fibrillation was defined as a ventricular rhythm with no recognizable QRS complex and with an amplitude less than that of the normal electrogram. In addition, the total time during which each heart remained in normal sinus rhythm within the first 5 minutes of reperfusion, was quantified.

Statistical Analysis

With the exceptions of incidences of arrhythmias (calculated in percentage of fibrillating hearts, reported to the total number of hearts in experiment), all the results are expressed as mean (±SEM).

2.5 In Vitro Antioxidant Capacity

Oxidation rate of N,N-diethyl-p-phenylenediamine (DPD) by a prooxidant system was used as a general reporter of the amount of ROS generated by that system (Anonymous, 1985; Chahine et al., 1991). Antioxidant capacity of preparations of TRIAD (or of its components) was defined as the extent (%) to which they inhibited the oxidation of DPD by prooxidants. To estimate the antioxidant capacity of TRIAD preparations in the conditions encountered during perfusion of hearts with electrolyzed buffer, 0.6 ml of modified KH not containing (control situation corresponding to 0% inhibition) or containing various concentrations of TRIAD, TRIAD (S2) or their components was subjected to 1-min electrolysis at 10 mA and then mixed with 0.3 ml of the non-electrolyzed counterpart of the solution to which 95 mM DPD was added. Determination of the amount of oxidized DPD was immediately done by reading absorbencies a 515 nm.

### 3. Results

3.1 Cardiac Own Effects of TRIAD and TRIAD (S2)

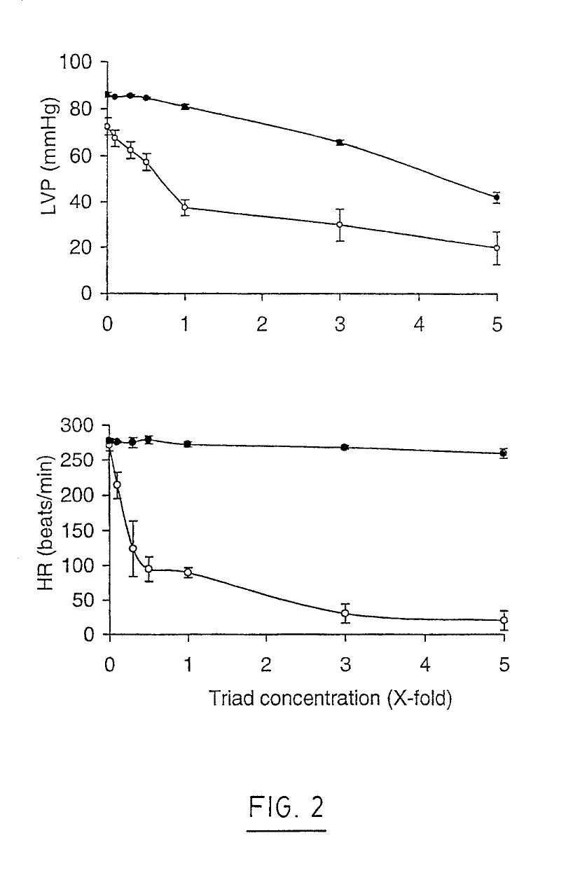

Genuine TRIAD preparations (prepared as Gokhale et al. (1997)) were detrimental to cardiac functions (FIG. 2), inducing a decrease in LVP and HR, even at low concentrations (less than 0.5×). The cardiotoxic effects observed with TRIAD on isolated heart, could probably be related to the fact that TRIAD preparation appears as a suspension, rather than a solution. This can mechanically affect function of the isolated heart which, when perfused with KH buffer only, does not benefit of the known tensioactive (detersive-like) effect of plasma components such as albumin. It is supposed that in vivo, such own effect of TRIAD will not occur. In contrast with the data on the cardiac function under TRIAD preparation, initial values of cardiodynamic variables were maintained when perfusion was done with TRIAD (S2) preparations (TRIAD previously centrifuged and filtered), for which low own cardiotoxic effects were found (FIG. 2). Although heart tolerance slightly dropped for concentrations of TRIAD (S2) higher than 1×, it was inconsequential for our studies since concentrations range equal to or lower than 1× were found to completely protect hearts as shown below.

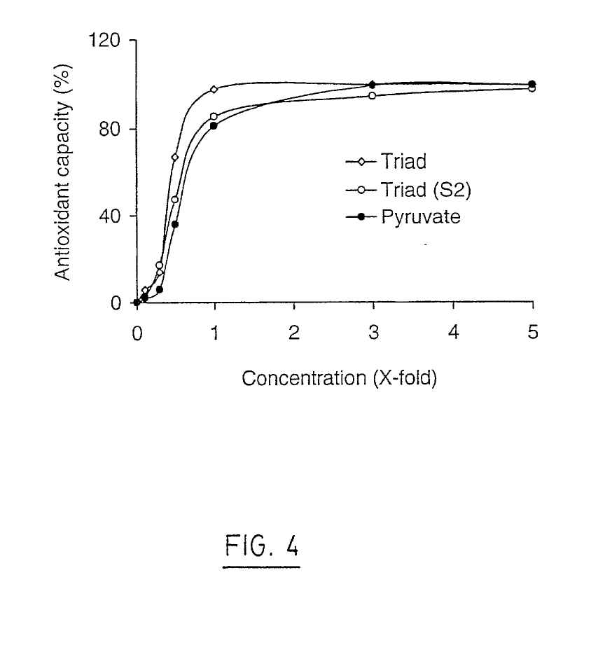

Furthermore, it is worth to mention that the antioxidant capacity of the TRIAD (S2) preparation did not differ from that of the standard TRIAD preparation (FIG. 4). This observation can be related to the fact that pyruvate (with a good aqueous solubility) seems to be responsible for most of the antioxidant capacities of TRIAD or TRIAD (S2) preparations (FIG. 4). In fact, pyruvate alone, at the same concentrations as in TRIAD and TRIAD (S2), exhibits only a slightly lower ROS scavenging capacity in vitro when compared to the whole TRIAD or TRIAD (S2) preparations. This can explain the relative similarity between the antioxidant behaviors of TRIAD and TRIAD (S2). Therefore, the S2 version of TRIAD preparations was used in heart perfusion studies.

However the results of FIG. 4 by no means indicate that pyruvate alone would be as efficient as TRIAD in heart model. In fact, the relative contribution of pyruvate and of TRIAD to heart protection when this organ is perfused with electrolyzed buffer or when it is submitted to ischemia-reperfusion it is still unknown. The relative response of TRIAD and of pyruvate alone likely depend of which reactive oxygen species are present in cells or organs. The ratios of reactive oxygen species such as ^{·}O₂⁻ (superoxide radical), H₂O₂ (hydrogen peroxide) and ^{·}OH (hydroxyl radical) are proned to continuous changes since they are affected by levels of antioxidant enzymes or molecules present inside and outside cells as well as levels of trace metal catalysts (such as Fe²⁺ ions). In addition, it is believed that individual contribution of TRIAD components to TRIAD effect will also change with duration of stress since repair mechanisms would become more essential after long periods of stress.

3.2 Cardioprotection Afforded by TRIAD Against Electrolysis-induced Oxidative Injury

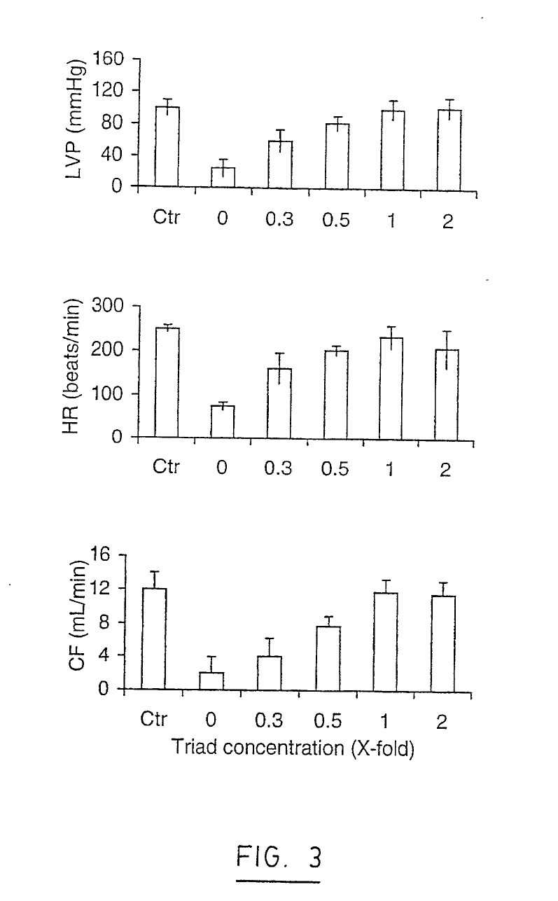

The concentration-related cardioprotection afforded by TRIAD in electrolysis is presented in FIG. 3. Electrolysis induced ROS generated important damages and dramatically decreased the level of all cardiodynamic variables (12% in case of CF, 18-20% for LVP and 30% for HR), in the absence (0×) of TRIAD (S2). A close to linear cardioprotection was established at increased TRIAD (S2) concentrations. Total cardiac recovery (100%), at the level of all variables (LVP, HR and CF), was found for concentrations 1× and above.

3.3 Cardioprotection Afforded by TRIAD Against Injury Induced by Ischemia-reperfusion

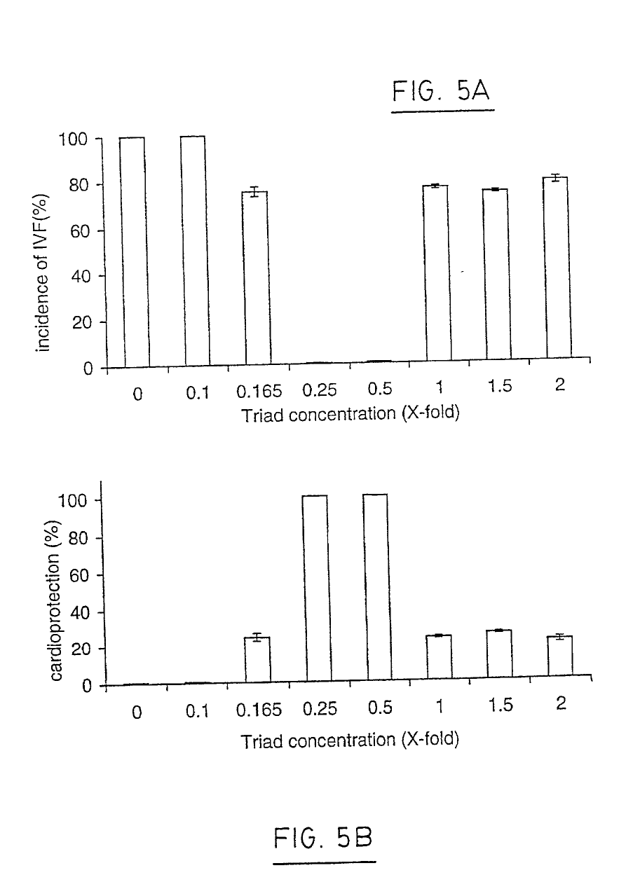

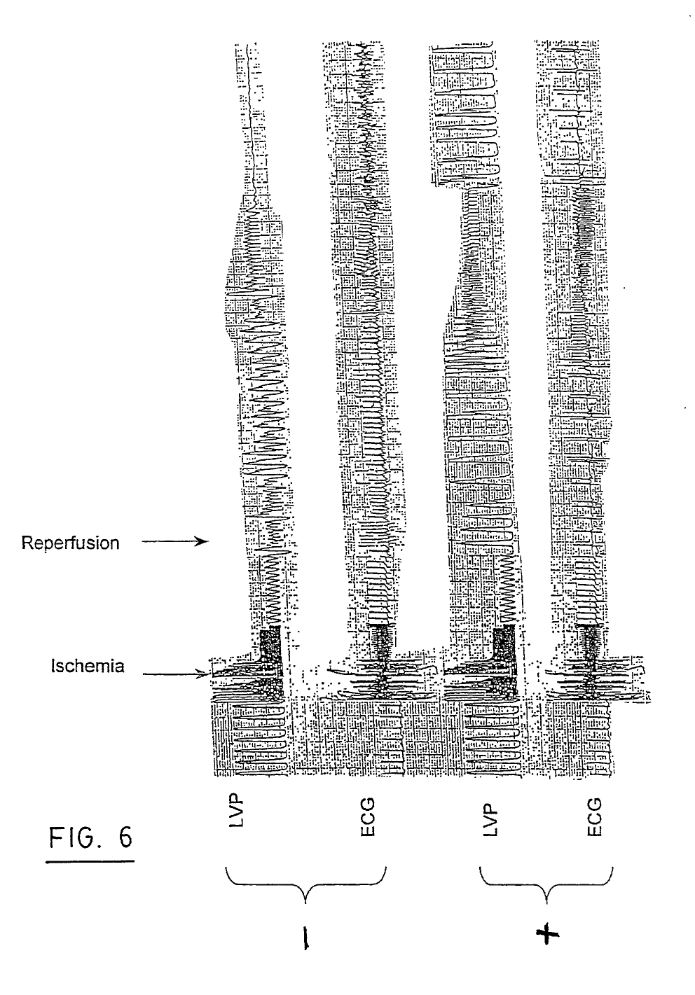

Reperfusion of ischemic hearts generates drastic damages. Control hearts (in the absence of cardioprotection) exhibited 100% irreversible fibrillation (over a period of more than 120 seconds; FIG. 5A). The total duration of normal sinus rhythm within 5 minutes of reperfusion was extremely short, only 25 sec. When injected to the perfusate, TRIAD in concentration of 0.25× and 0.50× totally reduced the incidence of reperfusion-induced irreversible ventricular fibrillations from 100% to 0% (FIG. 5B). The cardioprotection appears in a bell shaped concentration. Absence of TRIAD treatment resulted in an irreversible fibrillation and a total reduction of LVP with the heart arrest, while, under the TRIAD (S2) (0.5×), after a short period of fibrillation, the LVP is totally recovered (100%) and ECG returned to normal (FIG. 6).

Associated with the total elimination of the irreversible ventricular fibrillations (IVF) and with the decrease of duration of ventricular fibrillation, a large increase in the total duration of normal sinus rhythm was observed, in a concentration dependent manner, from 25 sec (without treatment) to more than 250 sec at reperfusion under TRIAD treatment.

The antiarrhythmic effect of TRIAD is concentration-dependent. Maximal antifibrillatory effects (0% IVT) and cardioprotection were observed for concentrations of 0.25-0.5× of TRIAD (S2) (FIGS. 5a and 5B). This bell-shaped dependency (FIG. 5B) of cardioprotection on the drug concentration is a quite general feature observed for many antifibrillatory agents [Atanasiu et al., 1995].

For comparison, we have examined the antiarrhythmic effects of deferoxamine (500 μM)—an iron chelator produced by bacteria (Streptomyces pilosus). TRIAD (S2) (0.25-0.5×) reduced the incidence of ventricular fibrillation to the same degree as Deferoxamine (500 μM) and as Ceruloplasmin 1 μM (Atanasiu et al, 1995). The incidence of irreversible ventricular fibrillation (IVF) was greatly diminished from 100% (untreated, control group) to 75% (with TRIAD 0.16×) and totally eliminated (0% IVF) under treatment with TRIAD 0.25-0.50×. Higher concentrations (1-2×) appear cardiotoxic in our experimental conditions (blood free perfusion). It is probably not the same in vivo, when plasma contributes with osmotic regulation of cardiac functions.

The results here reported are important, showing, for the first time, the cardioprotective and antifibrillatory effect of TRIAD on isolated heart. Under TRIAD cardioprotection, hearts totally recovered after ischemia and reperfusion, which represent events of high pathological risk.

### 4. Discussion

This study shows that TRIAD has an antioxidant protective action on isolated rat hearts exposed to oxidative stress, and results are summarized in Table I below.

The data obtained in this study clearly indicate the capacity of TRIAD to reduce significantly reperfusion-induced irreversible ventricular fibrillation in isolated rat heart Langendorff preparation.

During early reperfusion of ischemic myocardium, the sudden influx of oxygen in presence of metabolic intermediates accumulated during the ischemic period, will provide an ideal situation for the formation of OFR, exceeding the antioxidative capacity of the tissue. Oxygen free radicals, in particular the hydroxyl radical, may exacerbate ischemia induced injury by promoting oxidative modifications in cell membrane phospholipids, enzymes and ionic pumps. Altered electrophysiological membrane activity and calcium overload have been suggested as important factors underlying OFR-induced reperfusion arrhythmias.

TABLE I
Minimal concentration of TRIAD (X-fold) for complete antioxidant
protection.

| | Prooxidant system | |
|---|---|---|
| Model | Ischemia/reperfusion | Electrolysis |
| Heart (ex vivo) | 0.25 X | 1 X |
| | (Pyruvate, ↓) | (Pyruvate, ↑) |
| DPD (in vitro) | Not applicable | 1 X |
| | | (Pyruvate, ↑) |

The results are presented for heart-ex vivo model and for its in vitro counterpart (oxidation of DPD). TRIAD component that mostly contributed to overall antioxidant action is indicated between parentheses. Pyruvate action was apparently either decreased (↓) or increased (↑) by fatty acids and vitamin E. Electrolysis was tested with isolated hearts to compare the antioxidant action of TRIAD with that of ceruloplasmin.

For the cardioprotective effects of TRIAD it is supposed that the mechanism is related to its three components. Pyruvate, able to enter the cell, will enhance intracellular defense, while vitamin E and fatty acids will improve membrane functionality, eventually limiting the leakage of cellular Fe²⁺ ion (easily generated by reduction of Fe³⁺→Fe²⁺, induced by superoxide anion which is a reductive agent), preventing thus the production of hydroxyl radical (^{·}OH) via the Fenton and Haber-Weiss reactions,

Fenton reaction: Fe²⁺+H₂O₂→Fe³⁺+^{·}OH+OH⁻

Haber-Weiss reaction: Fe³⁺+^{·}O₂⁻→Fe²⁺+O₂

Mechanisms of iron involvement are not fully elucidated, but there is a growing consensus that oxidative tissue damage is related to non-heme cellular iron mobilized from cytosolic metal-containing sites: e.g. myoglobin and ferritin stores within endothelial and myocardial cells. Most of intracellular iron is deposited in ferritin (which can store 2000 up to 4500 of Fe³⁺ ions per complex) from where, in the presence of reducing equivalents (e.g. superoxide radicals), is released in the ferrous (Fe²⁺) form. This may explain the toxicity of superoxide anion. The initial damage results in a generalized release of iron into the cellular environment, and more widespread nonspecific injury may result. Although TRIAD and deferoxamine (iron-chelating agent) act by different mechanisms, their ultimate protective effects are probably exerted by the same prevention of ROS. Considering the low molecular weight of pyruvate and its easy access into the cell, TRIAD would be expected to intervene not only in the vascular space but also intracellularly. Thus, superoxide anions produced in endothelial cells at reperfusion may generate hydroxyl radicals via the iron-catalyzed Fenton reaction, damaging in this way the endothelium and adjacent contractile or conducting cells. For extracellular action of TRIAD in the case of intracellular OFR production, one should assume the outside diffusion of ferrous ions and of superoxide radicals. Post-ischemic reperfusion is often associated with the H₂O₂ release as a product of xanthine oxidase activity. Both superoxide anion and hydrogen peroxide have longer half-lives than the hydroxyl radical and can readily permeate cell membranes, either directly (H₂O₂) or through anion channels (superoxide radical). Since TRIAD was shown to decompose an important amount of H₂O₂ in vitro, the high cardioprotection found ex vivo, under TRIAD treatment, can fit with its action against H₂O₂ released in situ related to the oxidative damage. Thus, TRIAD can prevent hydroxyl radical formation from an intracellular source of superoxide radicals.

Protection of the myocardium against intracellular OFR can also be hypothetically explained by transcytosis of TRIAD (especially the easy access of pyruvate) from coronary capillaries into myocytes. Even high molecular weight molecules, as exogenous superoxide dismutase and catalase (240 kDa), after a brief episode of regional ischemia, were shown to be concentrated and transported across the capillary endothelium and into myocytes (Chudej et al., 1990).

Alternatively, the beneficial effects of TRIAD might be due to the prevention of hydroxyl radical generation from an extracellular source of superoxide production. In the isolated heart model, the only extracellular source of OFR production could be the autoxidation of catecholamines released from nerve endings, which accumulate in abnormal high concentrations in the ischemic myocardium. In a further work, we will try to establish if TRIAD can reduce the increase of noradrenaline efflux in the perfusate after electrolysis of perfusing buffer in isolated heart, suggesting a protection against free radical-induced injury to the sympathetic nerve endings.

It is worth to mention that no cardiotoxic effects were found with TRIAD S2 preparation, even at concentrations as high as 5×. TRIAD exhibits a concentration dependent cardioprotective effect in both electrolysis induced ROS and ischemia-reperfusion models. The cardioprotection is similar (although mechanisms are different) to that exerted by other cardioprotective agents as deferoxamine, ceruloplasmin, etc.

In conclusion, TRIAD exerts a strong antifibrillatory effect during reperfusion in the ischemic isolated rat heart, justifying its further consideration as a powerful protective agent against irreversible ventricular fibrillation, the most severe type of reperfusion-induced arrhythmias.

### 5. Conclusive Remarks

This study showed that TRIAD has an antioxidant cardioprotection on isolated rat hearts exposed to oxidative stress. Optimal concentrations vary with the type and prooxidant power of ROS generating systems. Pyruvate is a major contributor of antioxidant properties of TRIAD ex vivo and in cell cultures, especially when TRIAD is administered just prior induction of an oxidative stress and remains present for short time of hearts treatment (20-35 min). The contribution of vitamin E and egg yolk fatty acids may appear even more important in antioxidant defense when TRIAD is administered for longer periods (before, during and after oxidative stress). This study also yields in the development of an essential concept which comprises two aspects:

i) combinations of antioxidants having different mechanism of action provide higher protection to oxidative stress than any single antioxidant; and

ii) synergistic protection is a “latent” property of antioxidant combinations and does not necessarily manifest itself in all prooxidant conditions.

Finally, although the term “TRIAD” used herein refers to a composition comprising sodium pyruvate, vitamin E and egg yolk fatty acids, a person skilled in the art will understand that the composition of the present invention is not restricted to these sole specific components as explained previously in the first part of the section “DETAILED DESCRIPTION OF THE INVENTION”.

### 6. References

Throughout this paper, reference is made to a number of articles of scientific literature which are listed below:

Anonymous (1985) DPD calorimetric method. Standard methods for the examination of water and wastewater. New-York, APHA, AWWA, WPCF, 16^{th} ed., 306-309.

Atanasiu, R., Dumoulin, M. J., Chahine, R., Mateescu, M. A. and Nadeau, R. (1995) Can. J. Physiol. Pharmacol. 73, 1253-1261.

Chahine, R., Mateescu, M. A., Roger, S., Yamaguchi, N., De Champlain, J. and Nadeau, R. (1991) Can. J. Physiol. Pharmacol. 69, 1459-1464.

Chan, P. (1996) Stroke 27, 1124-1129.

Chudej L L, Koke J R, Bittar N. (1960) Cytobios 63, 41-53.

Ferdinandy P, Das D. K., Tosaki A (1993) J.Mol. Cell. Cardiol. 25, 683-692.

Gokhale, M. S., Lin, J. R. and Yager, J. D. (1997) Toxicol. in Vitro 11, 753-759.

Gutteridge, J. M. C. (1994) Annu. N.Y. Acad. Sci. 738, 201-213.

Jackson, C. V., Mickelson, J. K., Stringer, K., Rao, P. S., Lucchesi, B. R. (1986) J. Pharmacol. Methods 15, 305-320.

LeBel, C. P. and Bondy, S. C. (1991) Neurotox. Teratol. 13, 341-346.

Maiese, K. (1998) Clin. Neuropharmacol. 1, 1-17.

Martin, A. (1994) U.S. Pat. No. 5,926,370.

Martin, A. (1996) Dermatol. Surg. 22, 156-160.

Mateescu, M. A., Chahine, R., Roger, S., Atanasiu, R., Yamaguchi, N., Lalumière, G., Nadeau R., (1995) Arzneim. Forsch./Drug Res., 1995, 45, 476-80.

McCord J. M. (1985) N. Engl.J.Med. 312, 159-163.

Sheridan, J., Kern, E., Martin, A. and Booth, A. (1997) Antiviral Res. 36, 157-166.

Takemura, G., Onodera, T. and Ashraf, M. (1994) J. Mol. Cell Cardiol. 26, 41-454.

Vaughan, Williams (1991) Circulation 84, 1831-1851.

Walker, M. J. A., Curtis, M. J., Hearse, D. J., Campbell R. W. F., Janse, M. J., Yellon, D. M., Cobbe, S. M., Coker, S. J., Harness, J. B., Northover, B. J., Parratt, J. R., Riemersma, R. A., Riva, E., Russell, D. C., Sheridan, D. J., Winslow, E. and Woodward, B. (1988) Cardiovasc. Res. 22, 447.

Of course, numerous modifications and improvements could be made to the embodiments that have been disclosed herein above. These modifications and improvements should, therefore, be considered a part of the invention.

What is claimed is:

1. A method for treating a heart oxidative stress related condition, comprising administrating to a patient in need thereof a therapeutically effective amount of an antioxidative composition comprising pyruvate, at least one antioxidant and at least one lipid.

2. The method of claim 1, wherein said pyruvate, antioxidant and lipid are present in said composition in an amount that have a synergistic cardioprotective effect on cardiac cells.

3. The method of claim 1, wherein said at least one lipid consists of at least one fatty acid selected from the group consisting of monoglycerides, diglycerides triglycerides, free fatty acids, and mixtures thereof.

4. The method of claim 3, wherein said at least one fatty acid consist of a mixture of saturated and unsaturated fatty acids.

5. The method of claim 4, wherein said fatty acids are present in an amount varying from about 0.001% v/v to about 1% v/v, by weight of the composition.

6. The method of claim 1, wherein pyruvate is selected from the group consisting of pyruvic acid, pharmaceutically acceptable salts of pyruvic acid, prodrugs of pyruvic acid, and mixtures thereof.

7. The method of claim 1, wherein pyruvate is present in said composition in an amount varying from about 0.01 mM to about 20 mM.

8. The method of claim 1, wherein said at least one antioxidant is selected from lipid-soluble antioxidants.

9. The method of claim 1, wherein said at least one antioxidant is selected from the group consisting of Vitamin A, carotene, Vitamin E, pharmaceutically acceptable salts thereof, and mixtures thereof.

10. The method of claim 1, wherein said at least one antioxidant is selected from the group consisting of Vitamin E, Vitamin E acetate and analogues of Vitamin E.

11. The method of claim 1, wherein said at least one antioxidant is present in an amount varying from about 0.01 unit/ml to about 10 unit/ml of said composition.

12. The method of claim 1, wherein said composition further comprises an agent selected from the group consisting of metal chelators, metal scavengers, proteinic metal chelators, proteinic scavengers, preserving agents, solubilizing agents, stabilizing agents, wetting agents, emulsifiers, sweeteners, colorants, odorants, salts, buffers and coating agents.

13. The method of claim 1, for the treatment of heart attack/failure and the treatment of ischemic cardiopathy.

14. A method for treating a heart oxidative stress related condition comprising:
administrating, to a patient in need thereof, a therapeutically effective amount of an antioxidative composition comprising pyruvate and at least one antioxidant; and
providing into blood circulation of said patient at least one lipid having a synergistic therapeutic effect on cardiac cells in combination with said antioxidative composition.

15. The method of claim 14, wherein said at least one lipid is provided to said patient by increasing its lipidic blood level ratio through its diet.

16. The method of any one of claim 14, wherein said heart oxidative stress related condition is an heart attack/failure, ischemic cardiopathy, or handling an heart before and during an heart transplantation.

17. A method for the treatment of cardiac cells, comprising contacting said cells with a therapeutically effective amount of an antioxidative composition comprising pyruvate, at least one antioxidant and at least one lipid.

18. The method of claim 17, for protecting cardiac cells in vitro, in vivo and ex vivo against an oxidative stress related condition.

19. The method of claim 17, for the treatment of heart attack/failure, the treatment of ischemic cardiopathy, the conservation of heart before and during transplantation, and the treatment heart oxidative stress related condition(s).

20. A method for preparing a cardioprotective composition, comprising the steps of:
a) providing a therapeutically effective amount of: i) pyruvate, ii) at least one antioxidant; and iii) at least one lipid; and
b) mixing together the components i), ii) and iii) of step a) in a physiological buffered saline solution to obtain a pharmaceutically acceptable homologous suspension.

21. The method of claim 20, further comprising at least one of the steps of centrifuging or filtering the homologous suspension obtained in step b).
